(19) United States
(12) Patent Application Publication
(10) Pub. No.: US 2015/0056296 A1
(43) Pub. Date: Feb. 26, 2015

(54) KIT FOR PREPARING CARBON-DIOXIDE-CONTAINING COMPOSITION

(71) Applicant: Medion Research Laboratories Inc., Osaka (JP)

(72) Inventor: Masato Hiki, Osaka (JP)

(21) Appl. No.: 14/382,271

(22) PCT Filed: Feb. 26, 2013
(86) PCT No.: PCT/JP2013/054994

§ 371 Date: Aug. 29, 2014

(30) Foreign Application Priority Data
Feb. 29, 2012 (JP) 2012-044412

(51) Int. Cl. A61K 8/04; A61K 8/73; A61K 8/362; A61K 8/19

(52) U.S. Cl. CPC A61K 8/042; A61K 8/19; A61K 8/731; A61K 8/362; A61K 8/733; A61K 2800/10
USPC 424/613; 424/715

(57) ABSTRACT

[Problem] The objective of the present invention is, in a kit that is for preparing a carbon-dioxide-containing composition containing an oxygen-containing gel composition and a carbonate-containing gel composition, to provide a formulation technology enabling uniform and easy mixing, uniform generation of carbon dioxide, and the prevention of dripping down when applied to skin.

[Solution] In the kit for preparing a carbon-dioxide-containing composition, by means of selecting the combination of a gel composition (1) containing a hydrophobically modified alkyl cellulose, an acid, and water, and a gel composition (II) containing sodium alginate, a carbonate, and water, it is possible to obtain uniform mixing by means of a two-agent mixing device, uniform generation of carbon dioxide, and the prevention of dripping down when applied to skin.

### TECHNICAL FIELD

The present invention relates to a kit for preparing a carbon-dioxide-containing composition. More particularly, the present invention relates to a kit for preparing a carbon-dioxido-containing composition, which includes an acid-containing gel composition (I) and a carbonate-containing gel composition (II), wherein the gel compositions (I) and (II) can be easily mixed, and a carbon-dioxide-containing composition obtained by mixing the gel compositions (I) and (II) has such characteristics that carbon dioxide is uniformly generated, and dripping is hard to occur when the composition is applied to skin.

### BACKGROUND ART

In recent years, consciousness about cosmetic has been increased regardless of sex, and various functional cosmetics have been developed in response to this trend. Among them, cosmetics focusing on efficacy of carbon dioxide receive great attention. Carbon dioxide has been heretofore known to have a blood circulation-promoting effect, and it has been reported in Patent Document 1 that by using a carbon-dioxide-containing water-containing viscous composition with carbon dioxide efficiently confined therein, cosmetic effects such as improvement of skin quality, skin whitening, amelioration of flabbiness, amelioration of darkening and slimming, and therapeutic effects for skin disease/injury, dental disease, musculoskolotal disease and neurological disease are obtained.

Patent Document 1 discloses various forms of kits for obtaining a carbon-dioxide-containing composition with carbon dioxide efficiently confined therein. Above all, in Patent Document 1, a combination of a granule containing an acid and a water-containing viscous composition containing a carbonate is very effective for obtaining a carbon-dioxide-containing composition with carbon dioxide efficiently confined therein, and has been put to practical use.

As disclosed in Patent Document 1, a carbon-dioxide-containing composition can also be obtained from a combination of an acid-containing gel composition and a carbonate-containing gel composition. In the case of a combination of such gel compositions, the compositions can be easily and conveniently mixed using a two-agent mixing device (a device configured such that two agents are separately stored, and come into a mixed state when discharged to outside), so that handiness is improved, leading to enhancement of versatility.

However, a general thickener suffers from a reduction in thickening effect when used under an environment of strong acidity, and when an acid-containing gel composition and a carbonate-containing gel-composition are prepared using a general thickener, there are the following disadvantages: (1) it is difficult to mix the compositions using a two-agent mixing device; (2) generation of carbon dioxide in a carbon-dioxide-containing composition obtained by mixing the compositions becomes non-uniform; and (3) a carbon-dioxide-containing composition obtained by mixing the compositions easily drips from skin.

Thus, in conventional techniques, uniform mixing by a two-agent mixing device, uniform generation of carbon dioxide and prevention of dripping in application to skin cannot be achieved yet in a kit for preparing a carbon-dioxide-containing composition, which includes an acid-containing gel composition and a carbonate-containing gel composition.

### PRIOR ART DOCUMENT

### Patent Document

Patent Document 1: International Publication No. WO 99/24043

### SUMMARY OF THE INVENTION

### Problems to be Solved by the Invention

The object of the present invention is to provide a formulation technique enabling uniform and easy mixing, uniform generation of carbon dioxide, and prevention of dripping in application to skin in a kit for preparing a carbon-dioxide-containing composition, which includes an acid-containing gel composition and a carbonate-containing gel composition.

### Means for Solving the Problem

The present inventors have extensively conducted studies for solving the above-described problem, and resultantly found that uniform mixing by a two-agent mixing device, uniform generation of carbon dioxide, and prevention of dripping in application to skin can be achieved by selecting the combination of a gel composition (1) containing a hydrophobically modified alkyl cellulose, an acid and water, and a gel composition (II) containing alginic acid and/or a salt thereof, a carbonate and water in a kit for preparing a carbon-dioxide-containing composition. The present invention has been completed by further conducting studies based on the above-described findings.

That is, the present invention provides inventions having the aspect described below.

Item 1. A kit for preparing a carbon-dioxide-containing composition, including a gel composition (I) containing a hydrophobically modified alkyl cellulose, an acid and water, and a gel composition (II) containing alginic acid and/or a salt thereof, a carbonate and water, wherein the gel composition (I) and the gel composition (II) are mixed at the time of use.

Item 2. The kit according to item 1, wherein the hydrophobically modified alkyl cellulose is stearoxyhydroxypropylmethyl cellulose.

Item 3. The kit according to item 1 or 2, wherein the content of the hydrophobically modified alkyl cellulose contained in the gel composition (I) is 0.1 to 10% by weight.

Item 4. The kit according to any one of items 1 to 3, wherein the gel composition (II) further contains carrageenan and cellulose gum.

Item 5. The kit according any one of to items 1 or 4, wherein the content of alginic acid and/or a salt thereof contained in the gel composition (II) is 0.1 to 20% by weight.

Item 6. The kit according to any one of items 1 to 5, wherein the carbon-dioxide-containing composition is used as a cosmetic or an external pharmaceutical composition.

Item 7. A carbon-dioxide-containing composition being produced by the kit according to any one of items 1 to 6.

Item 8. The carbon-dioxide-containing composition according to item 7, wherein the composition is a cosmetic or an external pharmaceutical composition.

Item 9. A cosmetic method including a step of applying a carbon-dioxide-containing composition, the composition being obtained by mixing a gel composition (I) containing a hydrophobically modified alkyl cellulose, an acid and water and a gel composition (II) containing alginic acid and/or a salt thereof, a carbonate and water, to a body part of a person in need of cosmetic.

Item 10. Use of a gel composition (I) containing a hydrophobically modified alkyl cellulose, an acid and water and a gel composition (II) containing alginic acid and/or a salt thereof, a carbonate and water for production of a cosmetic.

### Advantages of the Invention

According to the present invention, an acid-containing gel composition and a carbonate-containing gel composition can be uniformly mixed by a two-agent mixing device, and as a result, a carbon-dioxide-containing composition with carbon dioxide generated uniformly can be obtained. Further, the carbon-dioxide-containing composition obtained according to the present invention is configured to be prevented from dripping when applied to skin, and has high handiness.

Moreover, according to the present invention, carbon dioxide can be efficiently confined in the carbon-dioxide-containing composition, and therefore various cosmetic effects and medical effects based on carbon dioxide can be effectively exhibited to skin to which the composition is applied.

### BRIEF DESCRIPTION OF THE DRAWINGS

FIG. 1 is a view showing a photograph of an external appearance of a Y-shaped two-agent mixing device used in the test example.

FIG. 2 is a view showing a result of observing a state of a mixture of gel compositions (I) and (II) (carbon-dioxide-containing composition) used in Example 1.

FIG. 3 is a view showing a result of observing a state of a mixture of gel compositions (I) and (II) (carbon-dioxide-containing composition) used in Comparative Example 2.

### EMBODIMENTS OF THE INVENTION

The present invention provides a kit for preparing a carbon-dioxide-containing composition, which includes a gel composition (I) containing a hydrophobically modified alkyl cellulose, an acid and water, and a gel composition (II) containing alginic acid and/or a salt thereof, a carbonate and water, wherein the gel composition (I) and the gel composition (II) are mixed at the time of use. Hereinafter, the kit of the present invention will be described in detail.

### Gel Composition (I)

The gel composition (I) contains a hydrophobically modified alkyl cellulose. The hydrophobically modified alkyl cellulose is a compound obtained by modifying a water-soluble cellulose ether derivative such as methyl cellulose, ethyl cellulose, propyl cellulose, butyl cellulose, hydroxyethyl cellulose, hydroxypropyl cellulose or hydroxypropylmethyl cellulose with a long-chain alkyl group, and specifically it is represented by the following general formula (1).

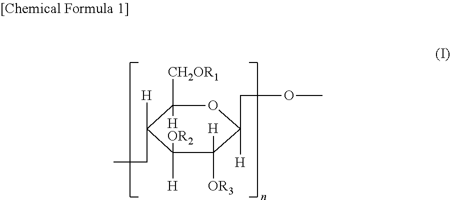

In the general formula (1), R₁, R₂ and R₃ are the same or different, and each represent at least one selected from —[CH₂CH(CH₃)O]ₓ—R₄, —[CH₂CH₂O]_{y}—R₄ and —[CH₂CH(OH)CH₂O]_{z}—R₄. Here, x, y and z are the same or different, and each represent an integer of 0 to 4.

R₄ represents at least one group selected from a hydrogen atom, a linear or branched alkyl group with a carbon number of 1 to 4, and a linear alkyl group with a carbon number of 10 to 28, and R₄ on at least one site in the general formula (1) is a linear alkyl group with a carbon number of 10 to 28. The linear alkyl group with a carbon number of 10 to 28 is preferably a linear alkyl group with a carbon number of 12 to 22.

In the general formula (1), n represent an integer of 200 to 200000.

In the present invention, the hydrophobically modified alkyl celluloses may be used alone, or may be used in combination of two or more thereof.

Among these hydrophobically modified alkyl celluloses, hydroxypropylmethyl cellulose containing a linear alkyl group with a carbon number of 10 to 28 is preferred, with stearoxyhydroxypropylmethyl cellulose being further preferred, from the viewpoint of further effectively achieving uniform and easy mixing of the gel composition (I) and the gel composition (II), uniform generation of carbon dioxide, and prevention of dripping in application to skin.

The hydrophobically modified alkyl cellulose for use in the present invention is preferably water-soluble.

When stearoxyhydroxypropylmethyl cellulose is used, a preferred example thereof is one in which among hydroxyl groups of a glucose residue as a constituent unit of the cellulose, 20 to 40%, preferably 27 to 30% of the hydroxyl groups are replaced by methoxyl groups; 5 to 20%, preferably 7 to 11% of the hydroxyl groups are replaced by hydroxypropoxyl groups; and 0.1 to 1.5%, preferably 0.3 to 6% of the hydroxyl groups are replaced by stearyloxyhydroxypropoxyl groups.

As the hydrophobically modified alkyl celluloses, one produced in accordance with a known production method may be used, or a commercial product may be used. For example, stearoxyhydroxypropylmethyl cellulose is commercially available as trade names “SANGELOSE 90L”, “SANGELOSE 90M” and “SANGELOSE 60L” (each manufactured by Daido Chemical Corporation).

While the content of the hydrophobically modified alkyl cellulose in the gel composition (I) is not particularly limited as long as it is in the form of a gel having viscosity, and the object of the present invention can be achieved, the content of the hydrophobically modified alkyl cellulose in the gel composition (I) is, for example, 0.1 to 10% by weight, preferably 0.5 to 5% by weight, further preferably 0.8 to 5% by weight, especially preferably 0.8 to 3% by weight.

The gel composition (I) contains an acid. The acid to be used in the gel composition (I) is not particularly limited as long as it is a water-soluble and pharmaceutically or fragrantly acceptable acid, and the acid may be an organic acid or an inorganic acid. Specific examples of the organic acid for use in the present invention include linear fatty acids such formic acid, acetic acid, propionic acid, butyric acid and valeric acid; dicarboxylic acids such as oxalic acid, malonic acid, succinic acid, glutaric acid, adipic acid, pimelic acid, fumaric acid, maleic acid, phthalic acid, isophthalic acid and terephthalic acid; acidic amino acids such as glutamic acid and aspartic acid; and oxy acids such as glycolic acid, malic acid, tartaric acid, citric acid, lactic acid, hydroxyacrylic acid, α-oxybutyric acid, glyceric acid, tartronic acid, salicylic acid, gallic acid, tropic acid, ascorbic acid and gluconic acid. Specific examples of the inorganic acid for use in the present invention include phosphoric acid, potassium dihydrogen phosphate, sodium dihydrogen phosphate, sodium sulfite, potassium sulfite, sodium pyrosulfite, potassium pyrosulfite, acidic sodium hexametaphosphate, acidic potassium hexametaphosphate, acidic sodium pyrophosphate, acidic potassium pyrophosphate and sulfamic acid. These acids may be used alone, or may be used in combination of two or more thereof.

Among these acids, citric acid, ascorbic acid and malic acid are preferred. Above all, acids related to in vivo TCA cycles (citric acid, malic acid and the like) acts to further enhance the cosmetic effect, and is suitably used in the present invention.

While the content of the acid in the gel composition (I) is appropriately set according to a type of the acid, an amount of the carbonate contained in the gel composition (II) described later, and so on, the content of the acid in the gel composition (1) is, for example, 0.1 to 10% by weight, preferably 0.1 to 5% by weight, further preferably 0.5 to 3% by weight.

The gel composition (I) contains water. Water to be used in the gel composition (I) may be, for example, purified water, distilled water, ion-exchanged water, tap water, ultrapure water, membrane-filtered water or the like.

While the content of water in the gel composition (I) is not particularly limited as long as the gel composition (I) can form a gel having moderate fluidity and viscosity, the content of water in the gel composition (I) is, for example, 70 to 99.9% by weight, preferably 80 to 99.5% by weight, further preferably 90 to 99.5% by weight.

The gel composition (I) may contain, in addition to the above-described components, other components capable of being blended in a cosmetic and an external pharmaceutical product as necessary. Examples of other components capable of being blended as described above include skin whitening agents, astringents, moisturizing agents, anti-inflammatory agents, ultraviolet absorbers, antiperspirants, plant extracts, amino acids, vitamins, alcohols, fat-and-oil surfactants, thickeners other than hydrophobically modified alkyl celluloses, antioxidants, chelating agents, antihistamines, sebum inhibitors, antiseborrheic agents, antipruritic agents, alcohols, fatty-and-oil components, surfactants, antioxidants, preservatives, germicides, chelating agents, flavors and pigments.

While the pH of the gel composition (I) is determined according to a type and an amount of the contained acid, the pH of the gel composition (I) is normally 1 to 6.5, preferably 1 to 5.5, further preferably 1.5 to 4.

When the hydrophobically modified alkyl cellulose coexists with water, the gel composition (I) is thickened to form a gel having moderate fluidity and viscosity. With a thickener other than a hydrophobically modified alkyl cellulose, the thickening effect is reduced under an environment of strong acidity formed by an acid dissolved in water, and therefore when a thickener other than a hydrophobically modified alkyl cellulose is mixed together with an acid and water, fluidity becomes excessively high, and viscosity becomes excessively low, so that the composition cannot be prepared in the form of a gel having moderate fluidity and viscosity. In contrast, the gel composition (I) can form a gel having moderate fluidity and viscosity to the extent that it can be used with a two-agent mixing device although the composition contains an acid because a hydrophobically modified alkyl cellulose is employed.

While the hardness of the gel composition (I) is not particularly limited, for example the hardness measured by a rheometer is normally 20 to 120 g, preferably 25 to 100 g, further preferably 25 to 70 g. Here, the “hardness measured by a rheometer” is a value obtained in the following manner: 50 g of a sample is added in a cylindrical container having a diameter of 3.6 cm and a depth of 4 cm, left standing at 20° C. for 12 hours or longer, and then measured at a needle insertion depth of 15 mm using a rheometer (1-CB-007 manufactured by Sun Scientific Co., Ltd.) with an adapter (disc having a diameter of 2 cm) set therein.

### Gel Composition (II)

The gel composition (II) contains alginic acid and/or a salt thereof as a thickener. In the present invention, the carbonate-containing gel composition (II) contains alginic acid and/or a salt thereof, and therefore when the gel composition (I) containing a hydrophobically modified alkyl cellulose and an acid and the gel composition (II) are mixed by a two-agent mixing device, the compositions can be mixed with high uniformity. As a result, a carbon-dioxide-containing composition with carbon dioxide generated uniformly can be obtained.

The alginic acid salt is not particularly limited as long as it is a pharmaceutically or fragrantly acceptable acid, and examples thereof include potassium alginate and sodium alginate, with sodium alginate being preferred. The content of alginic acid and/or a salt thereof in the gel composition (II) is not particularly limited, and is, for example, 0.1 to 20% by weight, preferably 0.5 to 6% by weight, further preferably 1 to 3% by weight.

The gel composition (II) may further contain other thickeners in addition to alginic acid and/or a salt thereof. The other thickener is not particularly limited as long as it is thickened in the presence of water, and the other thickener may be any of a natural polymer, a semisynthetic polymer, a synthetic polymer, an inorganic substance and so on.

Examples of the natural polymer to be used as the other thickener include plant-based polymers such as gum arabic, carrageenan, galactan, agar, quince seed, guar gum, gum tragacanth, pectin, mannan, locust bean gum, wheat starch, rice starch, corn starch and potato starch; microorganism-based polymers such as curdlan, xanthan gum, succinoglucan, dextran, hyaluronic acid and pullulan; and protein-based polymers such as albumin, casein, collagen, gelatin and fibroin.

Examples of the somisynthetic polymer to be used as the other thickener include cellulose-based polymers such as hydrophobically modified alkyl celluloses such as stearoxyhydroxypropylmethyl cellulose, ethyl cellulose, carboxymethyl cellulose and salts thereof (cellulose gum etc.), carboxymethylethyl cellulose and salts thereof, carboxymethyl starch and salts thereof, croscarmellose and salts thereof, crystalline cellulose, cellulose acetate, cellulose acetate phthalate, hydroxyethyl cellulose, hydroxypropyl cellulose, hydroxypropylmethyl cellulose, hydroxypropylmethyl cellulose phthalate, powdered cellulose, methyl cellulose and methylhydroxypropyl cellulose; starch-based polymers such as pregelatinized starch, partially pregelatinized starch, carboxymethyl starch, dextrin and methyl starch; alginic acid-based polymers such as alginic acid propylene glycol esters; and other polysaccharide-based polymers such as sodium chondroitin sulfate, sodium hyaluronate.

Examples of the synthetic polymer to be used as the other thickener include carboxyvinyl polymers, sodium polyacrylate, polyvinyl acetal diethyl aminoacetate, polyvinyl alcohol, polyvinyl pyrrolidone, methacrylic acid-ethyl acrylate copolymers, methacrylic acid-ethyl methacrylate copolymers, ethyl metacrylate/trimethyl ammonium ethyl chloride methacrylate copolymers and dimethylaminoethyl methacrylate/methyl methacrylate copolymers.

Examples of the inorganic substance to be used as the other thickener include water-containing silicon dioxide, light silicic anhydride, colloidal alumina, bentonite and laponite.

These other thickeners may be used alone, or may be used in combination of two or more thereof.

Among these other thickeners, stearoxyhydroxypropylmethyl cellulose, carrageenan and cellulose gum are preferred, with carrageenan and cellulose gum being further preferred, from the viewpoint of further effectively achieving uniform and easy mixing of the gel composition (I) and the gel composition (II), uniform generation of carbon dioxide, and prevention of dripping in application to skin.

A preferred aspect of the thickener to be used in the gel composition (II) is a combination of sodium alginate, carrageenan and cellulose gum. When these thickeners are used in combination, the ratio thereof is not particularly limited, but for example, 10 to 3000 parts by weight of carrageenan and 10 to 1000 parts by weight of cellulose gum, preferably 30 to 1000 parts by weight of carrageenan and 30 to 3000 parts by weight of cellulose gum are combined based on 100 parts by weight of sodium alginate.

The total content of thickeners in the gel composition (11) is, for example, 0.1 to 20% by weight, preferably 0.5 to 6% by weight, further preferably 1 to 3% by weight.

The gel composition (II) contains a carbonate. While the carbonate to be used in the gel composition (II) is not particularly limited as long as it is water-soluble, and can produce carbon dioxide by reacting with an acid in the presence of water, examples thereof include ammonium carbonate, potassium carbonate, sodium carbonate, sodium hydrogen carbonate, potassium sesquicarbonate, calcium sesquicarbonate, sodium sesquicarbonate and potassium hydrogen carbonate.

These carbonates may be used alone, or may be used in combination of two or more thereof.

Among these carbonates, sodium hydrogen carbonate is preferred.

While the content of the carbonate in the gel composition (II) is appropriately set according to a type of the carbonate, a type and amount of the acid contained in the gel composition (I) described above, and so on, the content of the carbonate in the composition (II) is, for example, 0.1 to 10% by weight, preferably 0.5 to 4% by weight, further preferably 1 to 2% by weight.

The gel composition (II) contains water. Water to be used in the gel composition (II) is similar to water in the gel composition (I) described above.

While the content of water in the gel composition (II) is not particularly limited as long as the gel composition (II) can form a gel having moderate fluidity and viscosity, the content of water in the gel composition (II) is, for example, 70 to 99.9% by weight, preferably 80 to 99.5% by weight, further preferably 90 to 99% by weight.

The gel composition (II) may contain, in addition to the above-described components, other components capable of being blended in a cosmetic and an external pharmaceutical product as necessary. Thus, other components capable of being blended are similar to those in the gel composition (I) described above.

While the pH of the gel composition (II) is determined according to a type and an amount of the contained acid, the pH of the gel composition (II) is normally 7.5 to 11, preferably 7.5 to 10, further preferably 8.0 to 9.5.

When alginic acid and/or a salt thereof coexist with water, the gel composition (II) is thickened to form a gel having moderate fluidity and viscosity.

While the hardness of the gel composition (I) is not particularly limited, for example, the hardness measured by a rheometer is normally 20 to 120 g, preferably 25 to 100 g, further preferably 25 to 70 g. Here, the “hardness measured by a rheometer” is as described in the foregoing section “Gel Composition (I)”.

### Use Mode

In the kit of the present invention, a carbon-dioxide-containing composition is prepared by mixing the gel compositions (I) and (II). When the gel compositions (I) and (II) are mixed, an acid contained in the gel composition (I) and a carbonate contained in the gel composition (II) react with each other to generate carbon dioxide, and the carbon dioxide is confined in a mixture of gel compositions (I) and (II) to form a carbon-dioxide-containing composition.

While the mixing ratio of the gel compositions (I) and (II) is appropriately set according to a composition of the gel compositions (I) and (II), but normally may be set such that the amount of the gel composition (II) is 50 to 200 parts by weight, preferably 75 to 150 parts by weight, further preferably 90 to 110 parts by weight based on 100 parts by weight of the gel composition (I).

The gel compositions (I) and (II) may be mixed by a spatula or the like with predetermined amounts of the gel compositions (I) and (II) placed in a container, but it is desirable to mix the compositions by a two-agent mixing device. The kit of the present invention enable uniform mixing of the gel compositions (I) and (I) oven when a two-agent mixing device is used, and carbon dioxide can be uniformly generated in the resulting carbon-dioxide-containing composition.

Here, the two-agent mixing device is a device configured such that the gel compositions (I) and (II) are separately stored, and the gel compositions (I) and (II) come into a mixed state when discharged to outside.

The two-agent mixing device is, for example, a two-agent mixing device including: a storage section 1 that stores the gel composition (I); a storage section 2 that stores the gel composition (II); a mixing section that mixes the gel compositions (I) and (II); a supply section 1 that supplies the gel composition (I) stored in the storage section 1 to the mixing section; a supply section 2 that supplies the gel composition (II) stored in the storage section 2 to the mixing section; and a discharge section that is coupled to the mixture to discharge a mixture of the gel composition (I) and the gel composition (II) to outside. Here, the mixing section may be provided with stirring means for stirring the supplied gel compositions (I) and (II), or the supply section 1 and the supply section 2 may communicate with each other to mix the gel compositions (I) and (II). Further, for example, the gel composition (I) stored in the storage section 1 and the gel composition (II) stored in the storage section 2 may be supplied to the mixing section under a pressure such as an external pressure to be mixed together.

Examples of two-agent mixing devices of other modes include a two-agent mixing device in which the gel composition (I) and the gel composition (II) are stored while being isolated from each other by an isolation section that can be collapsed under an external pressure or the like. In the two-agent mixing device, the gel compositions (I) and (II) are mixed by applying an external pressure or the like to collapse the isolation section at the time of use.

### Carbon-Dioxide-Containing Composition

The carbon-dioxide-containing composition obtained by the kit of the present invention is transdermally applied as cosmetics and external pharmaceutical compositions (including a quasi-drugs). The carbon-dioxide-containing composition obtained by the kit of the present invention can retain a high concentration of carbon dioxide, so that various cosmetic effects and medical effects based on carbon dioxide can be effectively exhibited. That is, the kit for preparing a carbon-dioxide-containing composition according to the present invention can be suitably used in cosmetic methods, methods for therapy of skin disease, and the like.

For example, when used as a cosmetic, the carbon-dioxide-containing composition is effective as a functional cosmetic intended for improvement of skin quality such as amelioration of rough skin; slimming of a body part such as a face, a leg, an arm, an abdomen, a flank, a back, a neck or a jaw (including amelioration of flabbiness, tightening of skin, etc.); or whitening, humidification, amelioration of darkening, amelioration of stains, amelioration of freckles, or the like.

When the carbon-dioxide-containing composition is used as a cosmetic, the carbon-dioxide-containing composition may be applied to a body part as an application object in a thickness of about 1 to 5 mm. While the application time in the case of use as a cosmetic may be appropriately set according to a use purpose, the carbon-dioxide-containing composition is applied, for example, for about 3 minutes to 1 hour per one time with a frequency of one time per 1 to 14 days.

For example, when used as an external pharmaceutical product, the carbon-dioxide-containing composition is effective as a prophylactic or therapeutic agent for itches associated with skin mucosa diseases or skin mucosa disorders such as athlete's foot, insect bite and sting, atopic dermatitis, nummular eczema, xeroderma, seborrheic dermatitis, hives, prurigo, housewives eczema, comedo, impetigo, folliculitis, carbuncle, furuncle, cellulitis, pyodermia, psoriasis, ichthyosis, palmoplantar hyperkeratosis, lichen, pityriasis, wound, heat burn, fissure, sore and frost bite; skin mucosa damages such as decubitus, wound, heat burn, angular cheilitis, skin ulcers, fissure, sore, frost bite and gangrene; engraftment failures of skin grafts, skin flaps and the like; dental diseases such as gingivitis, periodontitis, artificial tooth ulcers, blackening gingiva and stomatitis; skin ulcers, cold senses and numbness based on peripheral circulatory disturbances such as obstructive thromboangiitis, obstructive arteriosclerosis, diabetic peripheral circulatory disturbance and varicose vein of lower extremity; musculoskeletal diseases such as chronic rheumatoid arthritis, cervicobrachial syndrome, myalgia, arthralgia and lumbago; neurological diseases such as neuralgia, multiple neuritis and SMON disease; dyskeratoses such as psoriasis, corns, calluses, ichthyosis, palmoplantar hyperkeratosis, lichen and pityriasis; suppurative skin diseases such as comedo, impetigo, folliculitis, carbuncle, furuncle, cellists, pyodermia and suppurative eczema.

When the carbon-dioxide-containing composition is used as an external pharmaceutical composition, the carbon-dioxide-containing composition may be applied to an affected part of skin as an application object in a thickness of about 1 to 5 mm. While the application time in the case of use as an external pharmaceutical composition is appropriately set according to a use thereof, the carbon-dioxide-containing composition is applied, for example, for about 5 minutes to 24 hours per one time with a frequency of one time per 1 to 14 days.

### EXAMPLES

The present invention will be described in detail below by showing examples, but the present invention is not intended to be limited to these examples.

### Test Example

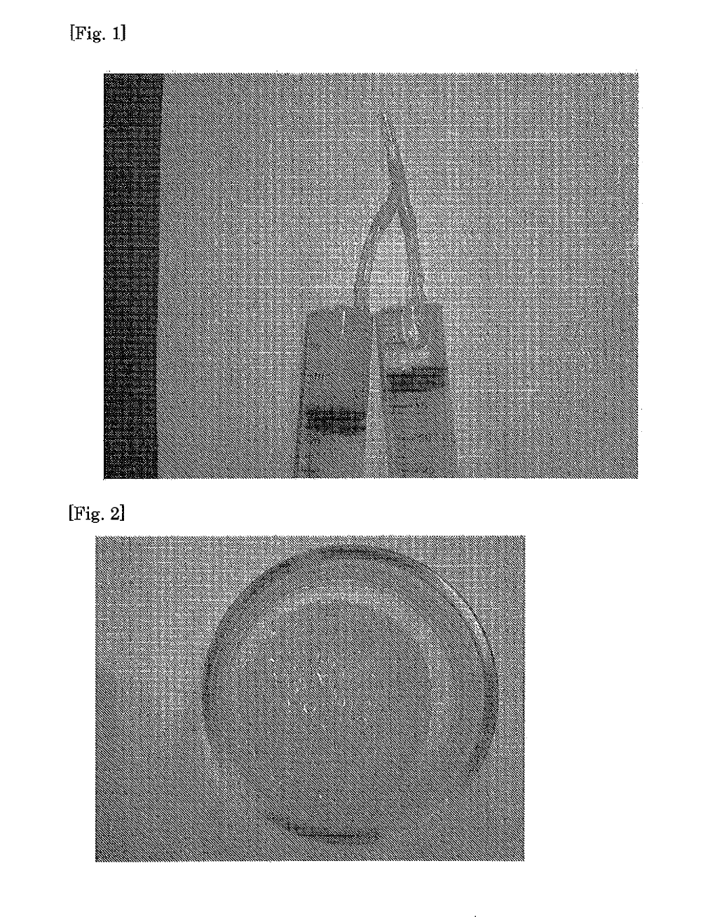

Gel compositions (I) and (II) having compositions shown in Tables 1 to 3 were prepared, and gel compositions (I) and (II) were mixed in amounts of 5 mL, respectively, using a Y-shaped two-agent mixing device. The Y-shaped two-agent mixing device used in this test is a device configured such that gel compositions (I) and (II) are each independently stored in each of two syringes (injection cylinders), supply lines for supplying gel compositions (I) and (II) are coupled to the two syringes, the supply lines coupled to the two syringes are coupled together into one line in some midpoint, so that gel compositions (I) and (II) are mixed and discharged to outside. FIG. 1 shows a photograph of the external appearance of the Y-shaped two-agent mixing device used in this test.

A mixture of gel compositions (I) and (II) (carbon-dioxide-containing composition) was discharged from the device, and external appearance properties (mixed state and foamability) were visually evaluated in accordance with the following criteria for the mixture (carbon-dioxide-containing composition) immediately after discharge and 10 minutes after discharge.

### <External Appearance Properties (Mixed State and Foamability)>

1: Gel compositions (I) and (II) are fully separated, and foaming is not observed.
2: Gel compositions (I) and (II) are almost separated, but are partially mixed together, and partial foaming is observed.
3: Gel compositions (I) and (II) are mixed together, but a color tone originating from the gel composition (I) and a color tone originating from the gel composition (II) are separated, and foaming is not observed.
4: Gel compositions (I) and (II) are mixed together, but a color tone originating from the gel composition (I) and a color tone originating from the gel composition (II) are separated, and foaming is not observed.
5: Gel compositions (I) and (II) are fully mixed together to form a color, and uniform forming is observed throughout the mixture.

Further, about 20 g of the mixture of gel compositions (I) and (II) (carbon-dioxide-containing composition) discharged from the device was applied to the face, and a state of the mixture (carbon-dioxide-containing composition) and a level of reddening of skin at an application site were visually evaluated in accordance with the following criteria. Reddening of skin at a applied site indicates that carbon dioxide was absorbed to promote blood circulation.

### <State of Mixture (Carbon-Dioxide-Containing Composition)>

0: An unresponsive area where carbon dioxide is not generated because mixing is not uniform is observed, and dripping occurs immediately.
1: An unresponsive area where carbon dioxide is not generated because mixing is not uniform is observed, and dripping occurs 5 minutes after application.
2: An unresponsive area where carbon dioxide is not generated because mixing is not uniform is observed, but dripping does not occur even 5 minutes after application.
3: An unresponsive area where carbon dioxide is not generated because mixing is not uniform is not observed, but dripping occurs immediately.
4: An unresponsive area where carbon dioxide is not generated because mixing is not uniform is not observed, but dripping occurs 5 minutes after application.
5: An unresponsive area where carbon dioxide is not generated because mixing is not uniform is not observed, and dripping does not occur even 5 minutes after application.

### <Level of Reddening of Skin at Application Site>

1: Skin is not reddened either 1 minute after application or 5 minutes after application.
2: Skin is slightly reddened 1 minute after application, but is not reddened 5 minutes after application.
3: Skin is slightly reddened both 1 minute after application and 5 minutes after application.
4: Skin is evidently reddened 1 minute after application, but is not reddened 5 minutes after application.
5: Skin is evidently reddened both 1 minute after application and 5 minutes after application.

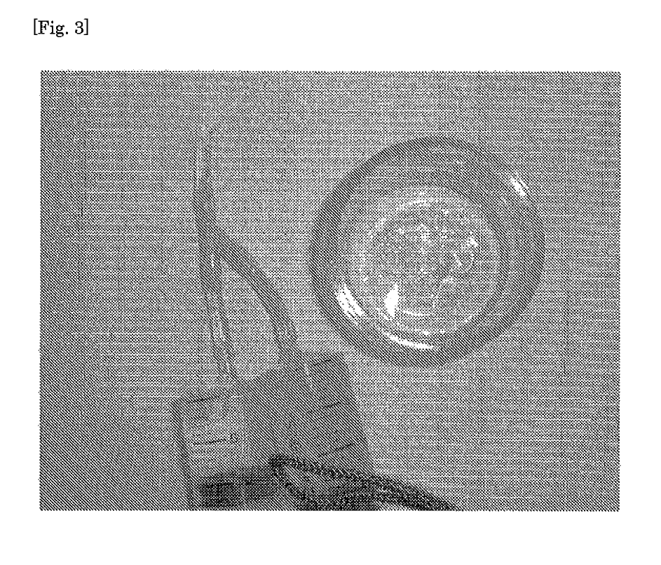

The obtained results are collectively shown in Tables 1 to 3. From these results, it has been found that in the case where stearoxyhydroxypropylmethyl cellulose was used as a thickener in the gel composition (1) containing an acid, and sodium alginate was used as a thickener in the gel composition (II) containing a carbonate, gel compositions (I) and (II) discharged from the device were uniformly mixed together, and carbon dioxide was uniformly generated. Further, it has become evident that in the case where stearoxyhydroxypropylmethyl cellulose was used as a thickener in the gel composition (1) containing an acid, and sodium alginate was used as a thickener in the gel composition (II) containing a carbonate, either formation of a lump or dripping did not occur when the carbon-dioxide-containing composition was applied to the face, and carbon dioxide was efficiently transdermally absorbed because sufficient reddening was observed. On the other hand, in the case where a thickener other than stearoxyhydroxypropylmethyl cellulose was used in the gel composition (I) containing an acid, or sodium alginate was not used as a thickener in the gel composition (II) containing a carbonate, dripping occurred when the carbon-dioxide-containing composition was applied to the face because gel compositions (I) and (II) discharged from the device were not uniformly mixed together, and reddening at the application site was insufficient. Gel compositions (I) and (II) used in Examples 1 and 2 both formed a gel having moderate viscosity. The mixture of gel compositions (I) and (II) (carbon-dioxide-containing composition) used in Examples 1 and 2 also formed a gel having moderate viscosity to the extent that the carbon-dioxide-containing composition was easily applied to skin. For reference, the result of observing a state of the mixture of gel compositions (I) and (II) (carbon-dioxide-containing composition) used in Example 1 is shown in FIG. 2, and the result of observing a state of the mixture of gel compositions (I) and (II) (carbon-dioxide-containing composition) used in Comparative Example 2 is shown in FIG. 3.

TABLE 1

| | | | Comparative Example | | | | | |
|---|---|---|---|---|---|---|---|---|
| | | Example 1 | 1 | 2 | 3 | 4 | 5 | 6 |
| Gel composition (I) | Citric acid | 1.0 | 1.0 | 1.0 | 1.0 | 1.0 | 1.0 | 1.0 |
| | Stearoxybydroxypropyl- | 1.0 | — | — | — | — | — | — |
| | methyl cellulose^{#1} | | | | | | | |
| | Sodium alginate | — | 0.5 | — | — | — | — | — |
| | Carrageenan | — | 2.0 | 2.0 | — | — | — | — |
| | Cellulose gum | — | 0.5 | — | 2.0 | — | — | — |
| | Xanthan gum | — | — | — | — | 1.0 | 1.0 | 0.5 |
| | Locust bean gum | — | — | — | — | — | 1.0 | — |
| | Water | Balance | Balance | Balance | Balance | Balance | Balance | Balance |
| | pH | 3.2 | Not | Not | Not | Not | Not. | Not |
| | | | measured | measured | measured | measured | measured | measured |
| Gel composition (II) | Sodium hydrogen | 1.5 | 1.5 | 1.5 | 1.5 | 1.5 | 1.5 | 1.5 |
| | carbonate | | | | | | | |
| | Sodium alginate | 0.5 | 0.5 | 0.5 | 0.5 | 0.5 | 0.5 | 0.5 |
| | Carrageenan | 2.0 | 2.0 | 2.0 | 2.0 | 2.0 | 2.0 | 2.0 |
| | Cellulose gum | 0.5 | 0.5 | 0.5 | 0.5 | 0.5 | 0.5 | 0.5 |
| | Water | Balance | Balance | Balance | Balance | Balance | Balance | Balance |
| | pH | 9 | Not | Not | Not | Not | Not | Not |
| | | | measured | measured | measured | measured | measured | measured |

| | | | | | | | |
|---|---|---|---|---|---|---|---|
| External appearance | 5 | 2 | 1 | 1 | 2 | 4 | 1 |
| properties of mixture | | | | | | | |
| discharged from device | | | | | | | |
| (Mixed state and foamability) | | | | | | | |
| (Immediately after discharge) | | | | | | | |
| External appearance | 6 | 2 | 1 | 1 | 2 | 4 | 1 |
| properties of mixture | | | | | | | |
| discharged from device | | | | | | | |
| (Mixed state and foamability) | | | | | | | |
| (10 minutes after discharge) | | | | | | | |
| State of mixture after | 5 | 0 | 0 | 0 | 0 | 2 | 0 |
| application to face | | | | | | | |
| Level of reddening of skin at | 5 | 2 | 1 | 2 | 2 | 2 | 2 |
| application site | | | | | | | |

In the table, the unit of the content of each blended component is “% by weight”.
^{#1}As stearoxyhydroxypropylmethyl cellulose, “SANGELOSE 60L” (trade name) (manufactured by Daido Chemical Corporation) was used.

TABLE 2

| | | Comparative Example | | |
|---|---|---|---|---|
| | | 7 | 8 | 9 |
| Gel | Citric acid | 1.0 | 1.0 | 1.0 |
| composition | (Acryloyl dimethyl taurine | — | — | 1.0 |
| (I) | ammonium/VP) copolymer | | | |
| | (Carboxyethyl acrylate/ | 1.5 | 3.0 | — |
| | acryloyl dimethyl taurine | | | |
| | Na) copolymer | | | |
| | Water | Balance | Balance | Balance |
| Gel | Sodium hydrogen carbonate | 1.5 | 1.5 | 1.5 |
| composition | Sodium alginate | 0.5 | 0.5 | 0.5 |
| (II) | Carrageenan | 2.0 | 2.0 | 2.0 |
| | Cellulose gum | 0.5 | 0.5 | 0.5 |
| | Water | Balance | Balance | Balance |

| | | | |
|---|---|---|---|
| External appearance properties of | 1 | 2 | 4 |
| mixture | | | |
| discharged from device | | | |
| (Mixed state and foamability) | | | |
| (Immediately after discharge) | | | |
| External appearance properties of | 1 | 2 | 4 |
| mixture | | | |
| discharged from device | | | |
| (Mixed state and foamability) | | | |
| (10 minutes after discharge) | | | |
| State of mixture after application to face | 0 | 2 | 2 |
| Level of reddening of skin at application | 2 | 3 | 4 |
| site | | | |

In the table, the unit of the content of each blended component is “% by weight”.

TABLE 3

| | | | Comparative |
|---|---|---|---|
| | | Example 2 | Example 10 |
| Gel | Citric acid | 1.0 | 1.0 |
| composition | Stearoxyhydroxypropylmethyl | 1.0 | 1.0 |
| (I) | cellolose^{#1} | | |
| | Water | Balance | Balance |
| Gel | Sodium hydrogen carbonate | 1.5 | 1.5 |
| composition | Stearoxyhydroxypropylmethyl | 1.0 | 1.0 |
| (II) | cellulose^{#1} | | |
| | Sodium alginate | 1.5 | — |
| | Water | Balance | Balance |

| | | |
|---|---|---|
| External appearance properties of mixture | 5 | 4 |
| discharged from device | | |
| (Mixed state and foamability) | | |
| (Immediately after discharge) | | |
| External appearance properties of mixture | 5 | 4 |
| discharged from device | | |
| (Mixed state and foamability) | | |
| (10 minutes after discharge) | | |
| State of mixture after application to face | 4 | 3 |
| Level of reddening of skin at application site | 4 | 3 |

In the table, the unit of the content of each blended component is “% by weight”.
^{#1}As stearoxyhydroxypropylmethyl cellulose, “SANGELOSE 60L” (trade name) (manufactured by Daido Chemical Corporation) was used.

1. A kit for preparing a carbon-dioxide-containing composition, comprising a gel composition (I) containing a hydrophobically modified alkyl cellulose, an acid and water, and a gel composition (II) containing alginic acid and/or a salt thereof, a carbonate and water, wherein the gel composition (I) and the gel composition (II) are mixed at the time of use.

2. The kit according to claim 1, wherein the hydrophobically modified alkyl cellulose is stearoxyhydroxypropylmethyl cellulose.

3. The kit according to claim 1, wherein the content of the hydrophobically modified alkyl cellulose contained in the gel composition (I) is 0.1 to 10% by weight.

4. The kit according to claim 1, wherein the gel composition (II) further contains carrageenan and cellulose gum.

5. The kit according to claim 1, wherein the content of alginic acid and/or a salt thereof contained in the gel composition (II) is 0.1 to 20% by weight.

6. The kit according to claim 1, wherein the carbon-dioxide-containing composition is used as a cosmetic or an external pharmaceutical composition.

7. A carbon-dioxide-containing composition being produced by the kit according to claim 1.

8. The carbon-dioxide-containing composition according to claim 7, wherein the composition is a cosmetic or an external pharmaceutical composition.

9. A cosmetic method including a step of applying a carbon-dioxide-containing composition, the composition being obtained by mixing a gel composition (I) containing a hydrophobically modified alkyl cellulose, an acid and water and a gel composition (II) containing alginic acid and/or a salt thereof, a carbonate and water, to a body part of a person in need of a cosmetic.

10. Use of a gel composition (I) containing a hydrophobically modified alkyl cellulose, an acid and water and a gel composition (II) containing alginic acid and/or a salt thereof, a carbonate and water for production of a cosmetic.
